(19) United States
(12) Patent Application Publication
(10) Pub. No.: US 2023/0132713 A1
(43) Pub. Date: May 4, 2023

(54) CATECHOL BASED HOT-MELT TISSUE ADHESIVE FOR HERNIA MESH REPAIR SURGERY

(71) Applicant: ISRAEL PLASTICS AND RUBBER CENTER LTD., Haifa (IL)

(72) Inventors: Dan LEWITUS, Herzliya (IL); Sabrina ROISMAN, Tel Aviv (IL); Anna DOTAN, Ramat Gan (IL)

(21) Appl. No.: 17/763,359

(22) PCT Filed: Sep. 24, 2020
(86) PCT No.: PCT/IL2020/051040

§ 371 Date: Mar. 24, 2022

Related U.S. Application Data
(60) Provisional application No. 62/906,400, filed on Sep. 26, 2019.

(51) Int. Cl. A61L 24/04; A61L 31/14; A61L 31/06; C08L 67/04; A61L 24/00

(52) U.S. Cl. CPC A61L 24/046; A61L 31/14; A61L 31/06; C08L 67/04; A61L 24/001

(57) ABSTRACT

There are provided herein bioadhesive compositions which include at least one catechol derivatives, such as, caffeic acid derivatives, and at least one synthetic thermoplastic polymer, wherein exposure to heat causes the bioadhesive composition to transform into a non-solid state and to cohesively adhere to a biological tissue upon subsequent cooling. Further provided are methods of making the compositions and uses thereof in repairing tissue damage, such as, for example, in hernia repair surgeries.

### TECHNICAL FIELD

The present disclosure relates generally to tissue adhesive compositions, methods for making the same and uses thereof in damaged tissue repair, such as, hernia repair surgery.

### BACKGROUND

Hernia repair surgery is one of the most common surgical procedures performed. Over 80% of hernia repairs performed in the US use mesh products. Even though the introduction of meshes for hernia repair has reduced the risk of chronic pain compared to suture repair by using tension free techniques, post-surgery chronic pain remains one of the main complications of mesh repair surgery. Chronic pain starting immediately after surgery is usually associated with nerve damage caused by sutures or tackers which are commonly used for the mesh fixation. To avoid these parenchyma-invasive techniques, which usually lead to secondary tissue damage, the use of tissue adhesives is of great interest.

Commonly used tissue adhesives suffer from several limitations, including, low adhesion strength under wet conditions, risks of virus transmission in biologically sourced adhesives (such as fibrin glues), cytotoxicity (for example, in cyanoacrylate based adhesives) and lack of specificity.

Mussel adhesive proteins (MAPs) have been suggested as potential alternative for tissue adhesives. These adhesive proteins, secreted by marine mussels, enable them to adhere to a wide range of underwater surfaces. Although the exact chemical adhesion mechanism is still unknown, MAPs are considered one of the most powerful adhesives, even stronger than other polymer based adhesives such as, epoxy and phenolic resins.

L-3,4-dihydroxyphenylalanine (DOPA) and its derivatives, such as 3,4-dihydroxyhydrocinnamic acid (HCA; hydrocaffeic acid) have been chemically coupled with synthetic and natural polymers to improve the efficacy of bio-adhesives presently used. Murphy et al. (2010), for example, presented a bio-adhesive based on the covalent bonding of HCA to polycaprolactone (PCL) and poly(ethylene glycol) (PEG).

Furthermore, it was reported that the incorporation of nano-particle sized LAPONITE® enhanced the mechanical properties of a similar tissue adhesive due to strong conjugation between the LAPONITE® and the catechol moieties of dopamine (Liu et. al., 2014). PEG was also used to increase the hydrophilicity of a PEG/silk fibroin adhesive hydrogel. The silk fibroin was chemically coupled with dopamine. The hydrogel showed improved water solubility and adhesion strength than the silk without catechol functionalization (Burke, et. al., 2015). It was also reported that the polar functional group of the catechol can be utilized to improve both chitosan solubility and bio-adhesive properties (Ryu, et. al., 2015). Fan et al. (2016) reported an aldehyde-free tissue adhesive by conjugating dopamine to a gelatin macromer intended for internal medical use. The adhesive was cross-linked by two cross-linking systems enhancing a rapid formation of wet adhesion and long-term resistance. Another research team developed a tissue adhesive for sternal bone closure based on the chemical modification of hyperbranched poly(β-amino ester) with poly(dopamine-co-acrylate). The adhesive has been reinforced with nanosized hydroxyapatite particles. The adhesion mechanism combined both the catechol moieties from the dopamine and the cross linkable vinyl functional groups leading to high mechanical strength, high wet adhesion properties and tailored curing speed by using different cross-linkers. Zhang et al. (2014) also co-polymerized dopamine with hyperbranched poly(β-amino ester) and poly(dopamine-co-acrylate).

However, the fabrication of bio-adhesives described above include chemical coupling of catechol moieties to a polymer, which usually requires complicated procedures and time-consuming processes. Additionally, adhesives based on mussel adhesive proteins show low cohesive strength, requiring the use of cross-linkers.

There is thus a need in the art for improved tissue adhesive compositions which are cost efficient, easy to apply and manufacture and which are capable of effectively, specifically and reversibly adhere to target tissues, under various conditions.

### SUMMARY

Aspects of the disclosure, according to some embodiments thereof, relate to catechol based hot-melt adhesive compositions which provide an “on-demand” and precise adhesion, along with rapid application and adequate adhesion strength for use, for example, in mesh fixation in hernia repair surgeries. In some embodiments, the thermal and rheological properties of the adhesive are particularly suitable for achieving low application temperature with optimum substrate wettability.

According to some embodiments there are provided tissue adhesive compositions capable of adhering to target tissues in an effective, specific, safe and reversible manner. In some embodiments, the bioadhesive compositions disclosed herein include at least caffeic acid derivatives, such as, HCA, and at least one polycaprolactone (PCL), such as, medium molecular weight PCL (mPCL).

According to some embodiments, there are provided advantageous bioadhesive compositions which include at least one caffeic acid derivative, at least one polycaprolactone, and optionally additional components, wherein the at least one caffeic acid derivative and the at least one polycaprolactone are not covalently bound thereto.

In some embodiments, the disclosed bioadhesive compositions are advantageous, as upon mild heating of the compositions, a hot-melt adhesive is obtained, to provide an on-demand adhesion, which is specific, precise, rapid, safe and cost efficient. Furthermore, as exemplified herein below, the disclosed bioadhesive compositions, provide adequate adhesion strength, for example, for use in mesh fixation in hernia repair surgeries. Additionally, as further exemplified herein below, in ex-vivo adhesion strength tests, the advantageous bioadhesive compositions disclosed herein exhibit outstanding adhesion strength compared to commercially available cyanoacrylate-based adhesive. For example, the hot-melt adhesive composition showed an increase of 29% in the adhesion strength, eight hours after its application on fresh abdominal wall porcine tissue.

According to some embodiments, without wishing to be bound to any theory or mechanism, due to the specific components of the compositions, their physical, thermal, mechanical and chemical properties, as well as the interplay there between, the bioadhesive composition act as a hot melt bio-adhesive that can be successfully applied to a biological soft tissue, whereby, it is heated to a fluid state, and once cooled on the tissue region it can cohesively adhere to the tissue. In some embodiments, the low melting temperature of the PCL (Tm of about 60° C.), when combined with the caffeic acid derivative, allows the composition to act as a hot melt bio-adhesive and be successfully applied to a biological soft tissue. The composition can be transformed to a non-solid (fluid) state by applying mild heat and once cooled, as it returns to its solid state, cohesively adhere to the tissue. In some embodiments, the compositions disclosed herein are further advantageous as they minimize the potential of tissue damage due to heat exposure because they allow a reduction in the bioadhesive compositions melting temperature. Furthermore, advantageously, the composition viscosity is maintained low enough to obtain a maximum wettability of the tissue to enhance adhesion. Thus, the thermal and rheological properties of the composition are particularly suited for use as a thermal-bioadhesive composition.

According to some embodiments, the disclosed bioadhesive compositions are further advantageous, as they are suitable for use as adhesive on a wet substrate.

According to some embodiments, as exemplified herein, HCA, a derivative catechol of caffeic acid, was used in the preparation of hot-melt bio-adhesive compositions together with medium weight PCL (mPCL). The adhesive compositions are prepared by melt-blending all the components, thereby avoiding the use of organic solvents. As exemplified herein, the incorporation of HCA not only decreased the activation temperature of the adhesive composition, but was also improved as compared to the adhesion strength of a cyanoacrylate based commercial tissue adhesive, in both poultry and porcine tissue ex-vivo experiments. In some embodiments, tertiary compositions have been prepared, aiming to further decrease the melting temperature of the adhesive, by including copolymer poly(trimethylene-co-polycaprolactone) (coPTMC) at various amounts in the compositions. Although coPTMC did not significantly decrease the melting temperature of the hot-melt adhesive, it caused a major impact on the melting enthalpy of the adhesive leading to up to 50% decrease. However, the coPTMC compromised the adhesion strength of the adhesive composition due to its high viscosity. Consequently, a quaternary tissue adhesive composition was designed, using a three-level full factorial design of experiment (DOE). As demonstrated herein, based on the DoE results, the complex viscosity of the adhesive had a major effect on the adhesion strength. The adhesive viscosity was decreased not only by the introduction of a lower molecular weight polymer (PCL14K), but also by increasing the temperature of application, which led to a higher adhesion strength and a shorter application time, while the maximum temperature measured on the tissue was not significantly increased. Furthermore, a 40:10 hot-melt adhesive composition exhibited burst strength values which are comparable to a commercial cyanoacrylate tissue adhesive, implying that the hot-melt adhesive compositions disclosed herein can function as suitable and advantageous soft tissue adhesives

In some embodiments, the bioadhesive compositions disclosed herein are particularly suitable for use in mesh fixation, for example, during hernia repair. Thus, the disclosed soft tissue bio-adhesive compositions may be utilized as an effective alternative for hernia mesh repair surgery, without causing further damage to the tissue.

According to some embodiments, there is provided a bio-adhesive composition, the composition comprising at least one catechol based components and at least one polycaprolactone. In some embodiments, the catechol based component is a caffeic acid derivative (such as, hydrocaffeic acid (HCA)), and the polycaprolactone is medium molecular weight PCL (mPCL). In some embodiments, the bioadhesive composition is a hot-melt composition, i.e., once heated it becomes at least partially fluid, and when cooled (for example after being applied to the target tissue), it can solidify and adhere to the tissue.

According to some embodiments, there is provided a bioadhesive composition which includes at least one caffeic acid derivative and at least one synthetic thermoplastic polymer, wherein exposure to heat causes the bioadhesive composition to transform into a non-solid state and to cohesively adhere to a biological tissue upon subsequent cooling thereof.

In some embodiments, the synthetic thermoplastic polymer includes polycaprolactone. In some embodiments, the polycaprolactone may include medium weight polycaprolactone. In some embodiments, the polycaprolactone may have a molecular weight of between about 20,000 Da to 90,000 Da.

In some embodiments, the bioadhesive composition may include at least about 15% (w/w) caffeic acid derivative of the total composition. In some embodiments, the composition may include about 15-25% w/w of caffeic acid derivative of the total composition. In some embodiments, the composition may include about 18-22% w/w of caffeic acid derivative of the total composition.

In some embodiments, the caffeic acid derivative may be hydroxy caffeic acid (HCA). In some embodiments, the caffeic acid derivative does not include an amine group.

According to some embodiments, the bioadhesive composition may further include copolymer poly(trimethylene-co-polycaprolactone) (coPTMC).

In some embodiments, the bioadhesive composition may further include polycaprolactone having a molecular weight in the range of about 5000-19,000 Da. In some embodiments, the additional polycaprolactone may have a MW of about 14,000 Da.

According to some embodiments, the bioadhesive composition may transform into a non-solid state at a temperature of over about 40° C. In some embodiments, the bioadhesive composition may transform into a non-solid state at a temperature in the range of between about 40° C. and 120° C., or any subranges thereof.

According to some embodiments, the bioadhesive composition may be in the form of a solid state film.

According to some embodiments, there is provide a method for producing the bioadhesive composition, the method includes mixing the caffeic acid derivative and the at least one synthetic thermoplastic polymer to form homogenous mixture while heating to a melting temperature.

According to some embodiments, the melting temperature is over about 100° C. In some embodiments, the melting temperature is over about 130° C.

According to some embodiments, the method may further include a step of cooling the composition to a solid state.

According to some embodiments, the method may further include a step of compression molding the composition to form a solid state film.

According to some embodiments, the bioadhesive composition is for use in adhering a substrate/matrix to a tissue. In some embodiments, the matrix may be selected from a mesh, gauze, dressing, bandage, and graft. In some embodiments, the mesh is a hernia mesh.

According to some embodiments, there is provided a kit comprising a substrate and the bioadhesive composition, wherein at least a portion of the substrate is coated, impregnated or formed with the bioadhesive composition. In some embodiments, the substrate in the kit may be selected from, but not limited to: a mesh, a gauze, a dressing, a bandage, a graft, or combinations thereof.

According to some embodiments, there is provided a method of placing the bioadhesive composition or the kit of onto or within a biological tissue region having a tissue damage or a region in predisposition of developing tissue damage; wherein placing of the bioadhesive composition or the kit includes allowing contact of the bioadhesive composition with the biological tissue region while the bioadhesive composition is heated to a temperature that causes the composition to be in a non-solid state and when in contact with the biological tissue region, allowing the bioadhesive composition to cool to the temperature of the biological tissue region, to thereby provide cohesive adherence of the bioadhesive composition or the bioadhesive kit to the biological tissue region.

According to some embodiments, placing includes holding the bioadhesive composition or kit in contact with the tissue region, until the bioadhesive composition is cooled to the tissue temperature.

According to some embodiments, the bioadhesive composition or bioadhesive kit may be heated immediately prior to, during, or after being in contact with the biological tissue region.

According to some embodiments, the method may include applying a continuous heat energy or pulsed heat energy to transform the bioadhesive composition into a non-solid state.

In some embodiments, the biological tissue region may be selected from the group of mucosal tissue and epithelial tissue.

In some embodiments, the biological tissue region may have a tissue damage selected from the group consisting of tissue protrusion, tissue weakening and tissue rupture. In some embodiments, the tissue damage may be caused by hernia surgery.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale. In the Figures:

FIG. 1 shows DSC thermogram of pristine hydrocaffeic acid;

FIG. 2—shows XRD pattern of HCA;

FIG. 3—shows XRD patterns for mPCL alone (“mPCL”) and mPCL with HCA (“mPCL:HCA”);

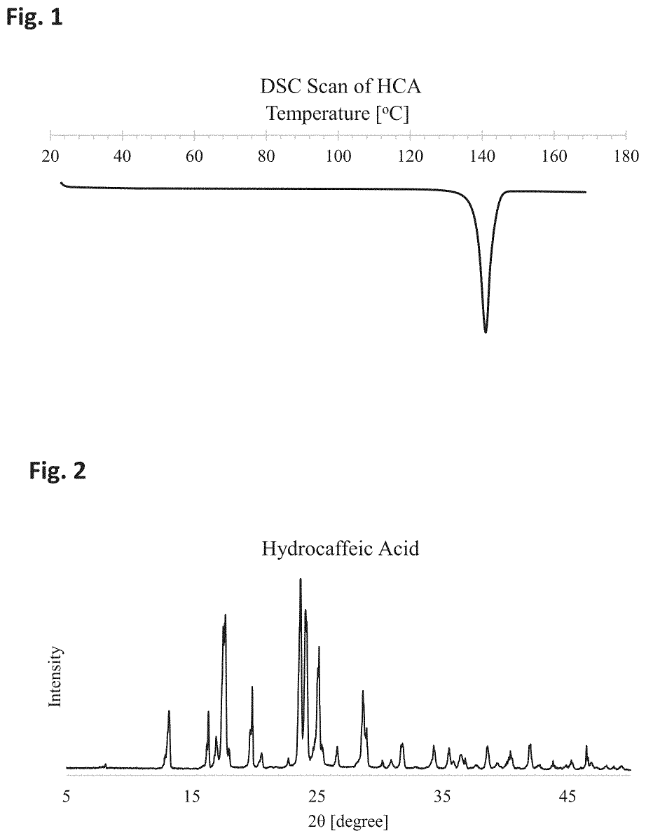

FIG. 1—Bar graphs showing results of lap shear adhesion test (kPa) on fresh poultry breast tissue, using the indicated compositions: mPCL (“mPCL neat” (mPCL without additives)), cyanoacrylate and composition of mPCL with HCA (“mPCL:HCA”);

FIG. 2—DSC thermograms of mPCL alone (“mPCL neat); mPCL with HCA “mPCL:HCA”) and composition of mPCL+HCA and further containing increasing amounts of coPTMC;

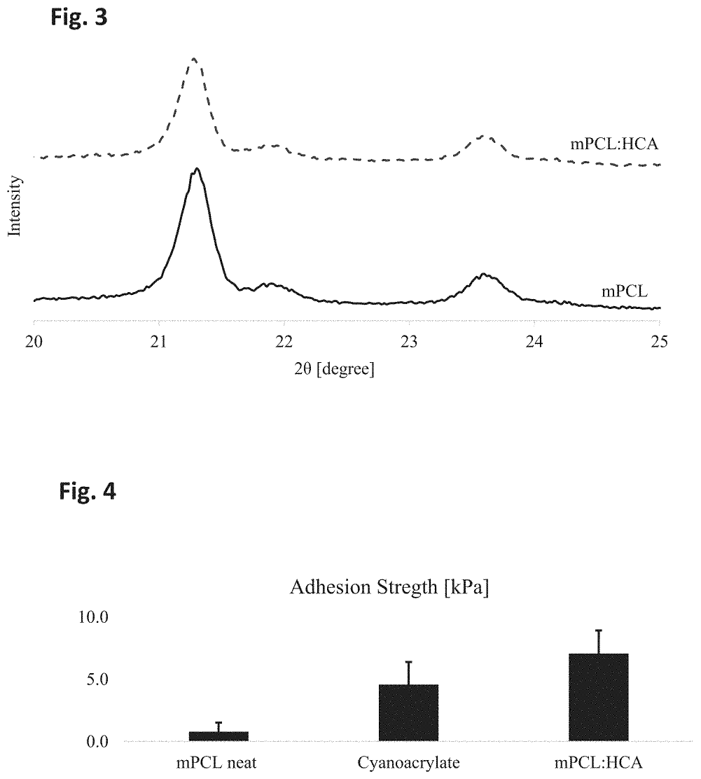

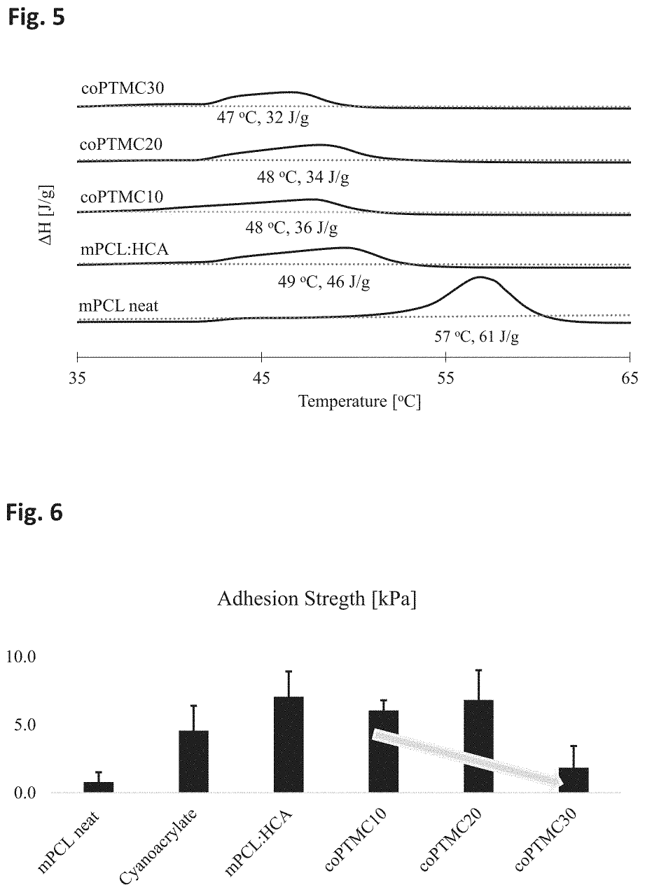

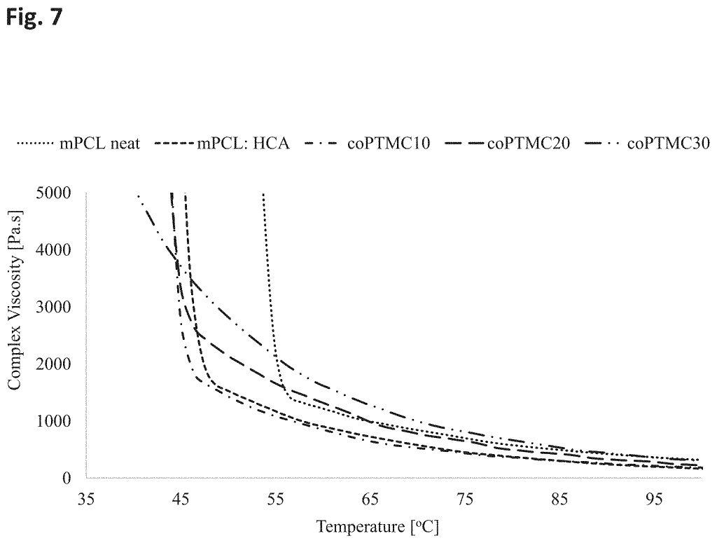

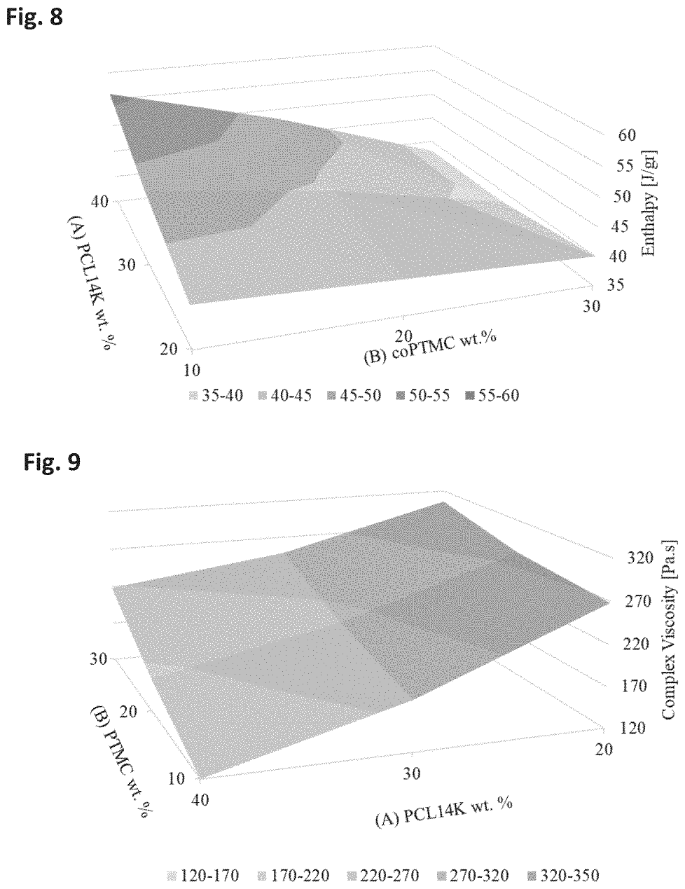

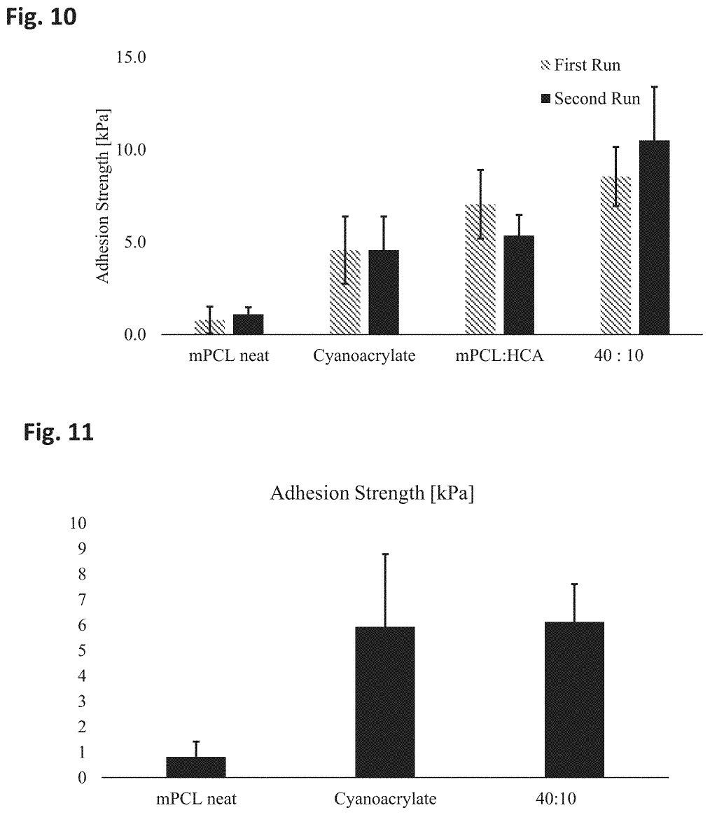

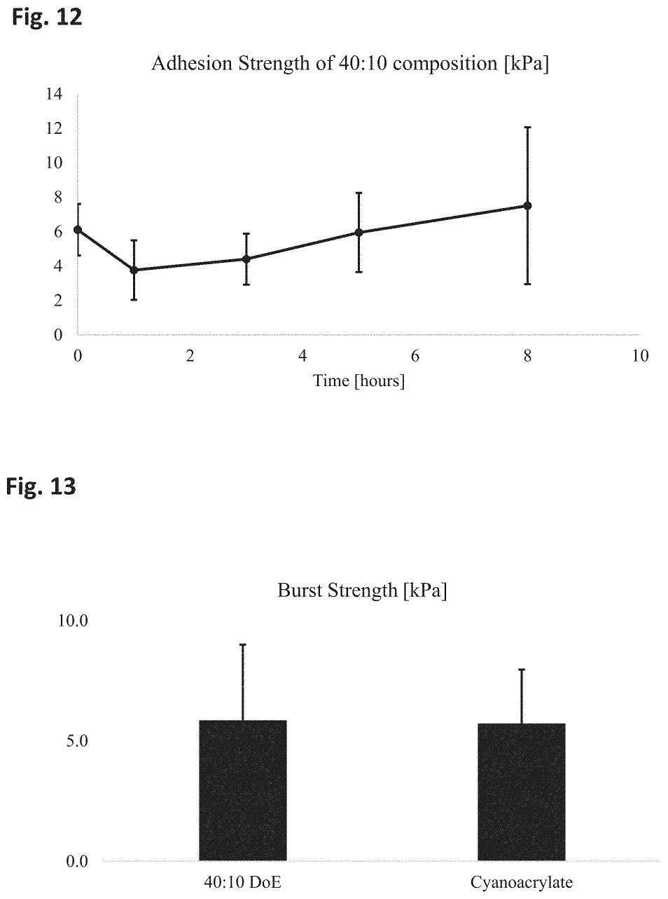

FIG. 3—Bar graphs showing results of lap shear adhesion test (kPa) on fresh poultry breast tissue, using the indicated compositions: mPCL alone (“mPCL neat”), composition of mPCL with HCA (“mPCL:HCA”) and compositions with increasing amounts of coPTMC. The arrow indicates a reduction in shear adhesion force between a composition including 10coPTMC and a composition including 30coPTMC;

FIG. 4—line graph showing the effect of inclusion of HCA and coPTMC in the indicated adhesive compositions on the complex viscosity (Pa·s) of the compositions, with increasing temperature (° C.);

FIG. 5—graphs showing 3D surface response relationship between components of the compositions, for enthalpy (ΔH).

FIG. 6—graphs showing 3D surface response relationship between components of the compositions, for polymer blend viscosity at 90° C.

FIG. 7—Bar graphs showing results of lap shear adhesion test (kPa) on fresh poultry breast tissue of the indicated compositions, using different parameters. In the first run (hatched bars), the adhesion test was performed at 70° C. for 20 seconds. In the first run (black bars), the adhesion test was performed at 95° C. for 5 seconds;

FIG. 8—Bar graphs showing adhesion strength (kPa) of the indicated compositions on fresh porcine abdominal wall tissue;

FIG. 9—Line graph showing adhesion strength (kPa) of an adhesive composition on fresh porcine abdominal wall tissue, as a function of time (hours).

FIG. 10—Bar graphs showing ball burst tests results of adhesive compositions, according to some embodiments, compared to a cyanoacrylate-based adhesive (Dermabond®).

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

The following are terms which are used throughout the description and which should be understood in accordance with the various embodiments to mean as follows:

As used herein, the term “bioadhesive composition” is used herein to denote an mixture of at least one thermoplastic polymer, being Polycaprolactone and at least one catechol derivative (such as, caffeic acid derivative), which under physiological environment is capable of stably adhering to biological tissue. Stable adherence as used herein, is directed to fixation to the tissue to an extent sufficient to receive the medical purpose of fixation. The fixation should be for a sufficient time and/or to a sufficient cohesive strength that allow the composition to remain connected and not tear apart from the tissue. It is appreciated that the higher the cohesive forces are, the stronger the adherence is. Thus, is some embodiments, the bioadhesive composition is at least partially cohesive, i.e. it may apply cohesive forces to the entire tissue surface in contact therewith, and/or it may contain areas of cohesive adherence to the surface. As exemplified herein, the level of adherence may be determined by various tests, such as peeling test, adhesion strength test, burst test, and the like.

In some embodiments, the adhesion to biological tissue is until the composition dissolves or disintegrates. In some embodiments, the adhesion to biological tissues may be for a time period sufficient to allow tissue regeneration and tissue growth at the area of damaged tissue.

According to some embodiments, a solid state and non-solid state of the compositions refers to the flowability of the composition or components thereof at room temperature (^{˜}25° C.). When in non-solid state, the composition flows to an extent sufficient for application (such as, spreading, pouring, introducing, or any other suitable application manner) of the composition into or onto the biological tissue. In some embodiments, flowability may be determined by any conventional liquid or fluid flow tests such as melt flow index (MFI), capillary rheometry. In some embodiments, flowability of the composition or components thereof may be characterized by its complex viscosity. In some embodiments, solidification includes transition into solid as well as semi solid form. These processes of melting and solidifying are reversible and may be repeated.

According to some embodiments, the composition includes at least one thermoplastic polymer. In some embodiments, the thermoplastic polymer is a polymeric matrix, which may include a single type of polymer, a combination of polymers or a combination of one or more polymers with other additives. In some embodiments, the thermoplastic polymer is a biocompatible polymer. In some embodiments, the thermoplastic polymer may be reversibly converted to a non-solid state when heated. When heated, it softens and fluidizes (melts) and once cooled, solidifies. In some embodiments, the melting point of the polymer may be in the range of about 50-180° C. In some embodiments, the melting point of the polymer may be in the range of about 50-120° C.

In some embodiments, the at least one thermoplastic polymer is polycaprolactone (PCL). In some embodiments, the PCL is a medium molecular weight PCL (mPCL). In some embodiments, the average MW of the PCL is in the range of about 20-90Kda. In some embodiments, the average MW of the PCL is in the range of about 30-70 kDa. In some embodiments the average MW of the PCL is in the range of about 40-50 kDa. In some exemplary embodiments, the average MW of the PCL is about 41,000 Da.

In some embodiments, the composition may include mPCL at about 40-80% of the total weight of the composition, or any subranges thereof. Each possibility is a separate embodiment. In some embodiments, the composition may include mPCL at about 10-40% of the total weight of the composition.

In some embodiments, the composition may further include an additional low molecular weight polycaprolactone. In some embodiments, low molecular weight (LMW) polycaprolactone has an average MW of about 5,000-19,000 Da. In some embodiments, the low molecular weight (LMW) polycaprolactone has an average MW of about 10,000-15,000 Da. In some exemplary embodiments, the average MW of the low molecular weight PCL is about 14,000 Da. In some embodiments, a low molecular weight PCL is about 14,000 Da having an average MW of about 14Kd is referred to herein as “PCL14K”).

In some embodiments, the compositions may include LMW polycaprolactone at about 10-40% w/w of the total weight of the composition, or any subranges thereof. Each possibility is a separate embodiment. In some embodiments, the compositions may include LMW polycaprolactone at about 20-30% w/w of the total weight of the composition.

In some embodiments, the composition may further include an additional polycaprolactone, such as, copolymer poly(trimethylene-co-polycaprolactone) (coPTMC). In some embodiments, the coPTMC is 9:1 copolymer poly(trimethylene-co-polycaprolactone).

In some embodiments, the compositions may include coPTMC at about 10-30% w/w of the total weight of the composition, or any subranges thereof. Each possibility is a separate embodiment.

According to some embodiments, the bioadhesive composition includes at least one catechol derivative. In some embodiments, the catechol derivative is a caffeic acid derivative. A caffeic acid is an organic compound that is classified as a hydroxycinnamic acid.

In some embodiments, a caffeic acid has the molecular structure of formula I:

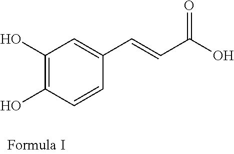 [SMILES: O=C(O)/C=C/c1ccc(O)c(O)c1]

In some embodiments, a caffeic acid derivative may be selected from, but not limited to: hydrocaffeic acid (HCA), 1-3,4-dihydroxyphenylalanine dopamine, dopamine, and the like.

In some embodiments, a hydrocaffeic acid (HCA) is 3,4-Dihydroxyhydrocinnamic acid, having the structure of Formula II:

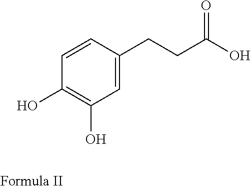 [SMILES: O=C(O)CCc1ccc(O)c(O)c1]

In some embodiments, the compositions includes Caffeic acid derivative at about 15-25% w/w of the total weight of the composition, or any subranges thereof.

In some embodiments, the ratio between the mPCL and the caffeic acid derivative in the composition is 4:1 (w/w), respectively.

The terms “Binary composition” and “Binary blend” may interchangeably be used. The terms refer to a bio adhesive composition, which includes at least two active components: a caffeic acid derivative and Polycaprolactone (PCL). In some exemplary embodiments, the binary composition includes HCA and mPCL.

The terms “tertiary composition” and “tertiary blend” may interchangeably be used. The terms refer to a bio adhesive composition, which includes at least two active components: a caffeic acid derivative and Polycaprolactone (PCL) and at least one additional component. In some embodiments, the tertiary composition is a binary composition which is added with copolymer poly(trimethylene-co-polycaprolactone) (coPTMC) at varying amounts.

The terms “quaternary composition” and “quaternary blend” may interchangeably be used. The terms refer to a bio adhesive composition, which includes at least two active components: a caffeic acid derivative and Polycaprolactone (PCL) and at least two additional components. In some embodiments, the quaternary composition is a tertiary composition which is further added with low molecular weight Polycaprolactone, such as, PCL14K.

According to some embodiments, the at least one thermoplastic polymer is water insoluble or insoluble in bodily fluid.

According to some embodiments, the composition may further include one or more additives, facilitating in the formation of the matrix, such as, plasticizers, surfactants, coupling agents, adhesion promoters, nucleating agents or fillers. The plasticizers may be used to reduce the brittleness and enhance flexibility of the bioadhesive composition. In some embodiments, the plasticizers may be at least one of tocopherol, tocopheryl-1polyethylene glycol succinate (TPGS), citrate plasticizer esters such as triethyl citrate, acetyl triethyl citrate, tributyl citrate, tributyl acetyl citrate (TAC), vitamin E (VI-E) or tri-(2-ethylhexyl)-citrate.

According to some embodiments, there is provided a method for preparing a bioadhesive composition comprising at least one caffeic acid derivative and at least one synthetic thermoplastic polymer, the method comprising mixing desired amounts of the caffeic acid derivative (such as, HCA) and the at least one synthetic thermoplastic polymer (such as, medium MW Polycaprolactone), to form homogenous mixture while heating to a melting temperature. In some embodiments, the method may further include a step of mixing one or more additional components, such as, LMW Polycaprolactone and/or coPTMC. According to some embodiments, the melting temperature is over about 100° C. In some embodiments, the melting temperature is over about 130° C.

According to some embodiments, the method for preparing a bioadhesive composition further includes a step of cooling the composition to a solid state.

In some embodiments, the method for producing the composition may further include a step of compression molding the composition to form a solid state film.

According to some embodiments, when making the bioadhesive composition, the conditions may be any that allow the formation of an essentially homogenous (typically flowing) mixture of the components. For example, the conditions may include mixing of a mixture at a temperature above the melting point of the thermoplastic polymer(s).

According to some embodiments, once an essentially homogenous mixture is formed, the composition may be allowed to cool to room temperature, or even below zero (for example, −20° C.), whereby a solid composite is formed. In some embodiments, Cooling may be on a support structure, in a mold or in an applicator. In some embodiments, the composition may heated and then compressed and molded to a desired for, such as, for example, a film. For example, the composition may be compression molded using a carver press operated at 85° C. for 5 min to form thin uniform films.

In some embodiments, cooling may be performed in mold, which provides laminates of the bioadhesive composition. In some embodiments, the mold is in a form allowing the formation of an essentially perforated solid structure, e.g. a net-like laminate. A perforated configuration is typically required when heating by diathermia. The laminate may be of any size or dimension, according to the particular need.

In some embodiments, the composition may be cooled in a mold, providing solid cylindrical sticks of the bioadhesive composition. Such sticks may be used with commercially available bioadhesive applicators. An applicator may include a housing, a heat sink and tip assembly and a cartridge assembly, the latter carrying the bioadhesive composition, and a plunger assembly for advancing the adhesive composition into the heat sink. The heat sink and tip assembly may be attached to the front of the housing for melting and dispensing the bioadhesive composition onto the surface area of the biological tissue in need thereof.

According to some embodiments, the bioadhesive composition may be cooled on a support structure/substrate/matrix or placed on the substrate at a later stage (e.g. prior to use). The association between the composition and the support structure may require re-heating of the composition. The support structure (substrate) may be any biocompatible material and at times a biodegradable material, for carrying the bioadhesive composition. Carrying may include any form of association between the support structure and the composition, including, impregnating or soaking the support structure with the composition (when the latter is in fluid form), coating (including extrusion coating) a surface of the support structure, spraying, lamination, layering over. The support structure may be in any form, including, for example, but not limited to: biocompatible synthetic fibers such as gauze, dressing, bandage, graft and mesh (such as those used in hernia), and the like, or combinations thereof.

In some embodiments, the substrate may be a polypropylene mesh. The polypropylene may be for example a monofilament polypropylene. In some embodiments, the polypropylene may be used in combination with additional materials such as, for example: poliglecarpone-25, oxidized regenerated cellulose, polytetrafluoroethylene (PTFE), expanded PTFE (ePTFE), silicon or polyvinylidene Fluoride (PVDF).

In some embodiments, the mesh may be made from other biocompatible materials such as prolene, surgipro, trelex, atrium, merselene polyglactin (vicryl) polyglycolic acid (dexon), polyester, polyethylene glycol, glycerol, PTFE or ePTFE or combinations thereof.

In some embodiments, the mesh may be made from polyester. In some exemplary embodiments, the substrate is made of polyethylene terephthalate.

In some embodiments, the combination of the substrate and the bioadhesive composition forms a bioadhesive kit. Upon exposure of the kit to heat (directly and/or indirectly (for example, by heating the tissue)), the associated bioadhesive composition provides medically stable (cohesive) fixation of the mesh onto the tissue on which it is placed.

In some embodiments, the bioadhesive composition and/or kit can be used for fixation onto bodily tissue, so as to repair tissue damage. The biological (bodily) tissue may be selected from, but not limited to: mucosal tissue, epithelial tissue, connective tissue, muscle tissue, blood vessel tissue (such as, endothelium tissue) and nervous tissue. In some exemplary embodiments, the biological tissue is a mucous or epithelial tissue. In some embodiments, the bodily tissue is a tissue region having a tissue damage. In some embodiments, the tissue damage may be selected from tissue protrusion, tissue weakening, tissue rupture, and the like, or any combinations thereof.

According to some embodiments, a tissue region may be any region of a biological tissue as defined hereinabove, possibly containing a damaged area or an area in predisposition of being ruptured or otherwise damaged or defected. Predisposition may be based on early prognosis, genetic tendency, medical history, and the like.

According to some embodiments, a “tissue damage” or “tissue defect” may include any type of damage to a tissue that may include, for example, tissue protrusion, tissue deformation, tissue weakening, tissue hole, tissue rupture, and the like. The tissue defect may occur in any anatomical portion of the body.

In some embodiments, exemplary embodiments, the tissue damage is a hernia, including various types of hernia, such as, but not limited to, Inguinal hernia, femoral hernia, umbilical hernia, incisional hernia, diaphragmatic hernia (hiatus hernia), congenital diaphragmatic hernia, Morgagni's hernia, Cooper's hernia, epigastric hernia: Littre's hernia: Lumbar hernia, Petit's hernia, Grynfeltt's hernia, Maydl hernia, Obturator hernia, Pantaloon hernia, Paraesophageal hernia, Paraumbilical hernia, Perineal hernia, Properitoneal hernia, Richter's hernia, Sliding hernia, Sciatic hernia, Spigelian hernia, sports hernia, Velpeau hernia, Amyand's hernia, and the like.

In some embodiments, the tissue may be a blood vessel tissue or external (for example, skin) wound. According to this embodiment, the tissue damage or tissue defect may be related to bleeding of blood vessels for example small blood vessels.

According to some embodiments, upon placing the composition or the kit onto the tissue region, they may be exposed to heat energy. In this context, “heating” or “exposure to heat energy” is used to denote the application of thermal heat or high frequency electromagnetic currents.

In some embodiments, heat may be generated from different types of energy, such as, for example, electrical energy, mechanical energy, chemical energy, nuclear energy, sound energy and thermal energy itself which are converted to heat energy. In some embodiments, the heat may be obtained using alternating current (AC) or direct current (DC). Heat may also be generated using ultrasonic or radiofrequency devices that are used to heat a thin wire (filament).

According to some embodiments, the heat may be applied onto the bioadhesive composition or onto the bioadhesive kit or onto the tissue region being in contact with the composition or kit, all of which eventually causing heating of the bioadhesive composition. The heat energy may be applied such as to be insufficiently intense to destroy tissue or to impair tissue vitality, but sufficient to cause the at least partial fluidizing of the bioadhesive composition which eventually results in adherence of the composition or kit to the tissue region. As an example, the exposure to heat energy is by heat conduction, e.g. from a metal probe heated by electric current.

In some embodiments, heat energy may be applied on at least one location (portion) of the bioadhesive composition or of the bioadhesive device (direct heating of the bioadhesive composition/device) or of the tissue region or of the surroundings (indirect heating of the composition/device).

According to some embodiments, placing the bioadhesive composition or kit may comprise holding the bioadhesive composition directly in contact with the tissue region while applying heat.

In some embodiments, the heat may be applied on or to the tissue region which in turn heats the bioadhesive composition. Alternatively, heating may include direct heating of the bioadhesive composition. Heating may be continuous or pulsed heating.

In some embodiments, the application of heat may be achieved by using dielectric energy applied on the tissue region. According to some other embodiments, application of heat energy may be by the use of electrocautery devices, (such as diathermy device).

In some embodiments, heating may be achieved, for example, using a hot plate, a harmonic heating device, bipolar surgical devices, an applicator in the form of a hot glue gun as well as any other device creating direct or residual heat.

According to some embodiments, upon heating, at least a portion of the bioadhesive composition melts and at least partially migrates towards the biological tissue, at which point, it is then cooled again to form the cohesive fixation effect.

According to some embodiments, specifically for hernia, the therapeutic method of using the bioadhesive composition may provide the surgeon with an improved attachment of a support structure such as a mesh or a graft on a damaged tissue region by an application of heat energy without applying mechanical force.

According to some embodiments, there is provided a kit which includes a substrate/support/matrix and the bioadhesive composition disclosed herein, wherein at least a portion of the substrate is coated, impregnated, soaked or formed with the bioadhesive composition. In some embodiments, the substrate is selected from a mesh, a gauze, a dressing, a bandage, a graft, or combinations thereof. In some embodiments, the kit is configured to be placed on a tissue, such as, a damaged tissue. In some embodiments, upon heating the kit (before, during or after being placed on the tissue region), when the kit is cooled after it is on the tissue region, the substrate may adhere to the tissue region.

According to some embodiments, there is provided a therapeutic method of placing a bioadhesive composition comprising at least one caffeic acid derivative and at least one synthetic thermoplastic polymer, or the bioadhesive kit onto or within a biological tissue region comprising a tissue damage or a region in predisposition of developing tissue damage; wherein placing of the bioadhesive composition or the kit comprises allowing contact of the bioadhesive composition with the biological tissue region while the bioadhesive composition is heated to a temperature that causes the composition to be in a non-solid state and when in contact with the biological tissue region, allowing the bioadhesive composition to cool to the temperature of the biological tissue region, to thereby provide cohesive adherence of the bioadhesive composition or the bioadhesive kit to the biological tissue region.

In some embodiments, placing includes holding the bioadhesive composition or the bioadhesive kit in contact with the tissue region, until the bioadhesive composition is cooled to the tissue temperature.

In some embodiments, the bioadhesive composition or bioadhesive kit is heated immediately prior to, during, or after being in contact with the biological tissue region.

In some embodiments, the method of placing may include applying a continuous heat energy or pulsed heat energy to transform the bioadhesive composition into a non-solid state.

According to some embodiments, the bioadhesive composition is for use in adhering a substrate/matrix/support to a tissue. In some embodiments, the matrix may be selected from a mesh, gauze, dressing, bandage, and graft. In some embodiments, the mesh is a hernia mesh.

According to some embodiments, the substrate/matrix/support adhered to tissue by the bioadhesive composition may be removed from the tissue, for example, by re-heating. For example, a care giver may heat the adhered matrix from the tissue to allow releasing and removing from the tissue, without harming or otherwise affecting the tissue. In some embodiments, the matrix and/or the bioadhesive composition may be removed from a tissue, after it has been applied thereto, by heating.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Materials and Methods

Polycaprolactone Mw=41,000 Da (Capa™ 6400, mPCL) was obtained from Perstop©. Hydrocaffeic acid (HCA) (3,4-dihydroxyhydrocinnamic acid), polycaprolactone Mw=14,000 Da (PCL14K), copolymer poly(trimethylene-co-polycaprolactone) (9:1) (coPTMC), Dulbeco's Phosphate Buffered Saline (DPBS, modified without calcium chloride and magnesium chloride) were purchased from Sigma Aldrich, Israel. Fresh poultry breast tissue was purchased from the market. Fresh porcine abdominal wall was purchased from Biotech Farm LTD, Israel. Parietex™ Hydrophilic 3-Dimensional Mesh from COVIDIEN™ was purchase from Medtronic, Israel.

### Adhesive Blends (Composition) Preparation and Characterization

### Adhesive Preparation

All blends have been prepared by simply melt blending all components to form homogenous blends in poly (tetrafluoro ethylene) (PTFE) cups on a hot plate set to 150° C. The blends were store at (−) 20° C. and then they were compression molded using a carver press operated at 85° C. for 5 min to form thin uniform films. Films were stored at (−) 20° C. prior to characterization. The polymer (Polycaprolactone):catechol ratio was 4:1 (w/w) and it was kept constant for all blends. For example, a Polycaprolactone:catechol ratio is 80%:20% w/w. In the polymer phase it may contain coPTMC and low molecular weight PCL.

For the binary blends, mPCL was blended with HCA. To the polymer phase, 10-30 wt % of coPTMC was introduced to obtain ternary blends. For the quaternary blends, nine blends were prepared: PCL14K low level, middle level and high level were 20, 30 and 40 wt %, respectively. coPTMC low level, middle level and high level were 10, 20 and 30 wt %, respectively.

### Thermal Analysis

Melting temperature (Tm) and melting enthalpy (ΔH) of the samples were tested using a TA Differential Scanning calorimeter (DSC) Q200 operated between −40° C. and 100° C. at a heating rate of 10° C./min with two heating runs.

### Crystalline Structure Analysis

Degree of crystallinity was calculated using the DSC results previously described, where each PCL blend was analyzed by the following equation:

Equation 1—Degree of crystallinity.

χ_%=(ΔH_m)/(% wt*ΔH_m\hat{ }o)

where χ_% is the degree of crystallinity of the PCL weight fraction (% wt) in the blend. ΔH_m\hat{ }o is the enthalpy of fusion of a 100% crystalline PCL with a value of 135.5 J/gr. In addition, X-ray diffraction (XRD) measurements were performed using a Rigaku SmartLab X-ray diffractometer with Cu-Kα radiation (λ=1.542 Å, 40 kV and 30 mA) in Bragg-Brentano geometry, in the 2θ range from 5° to 50°, with a step size of 0.02°, at 4°/min rate.

### Rheological Analysis

The rheological behavior of the indicated compositions was characterized using an oscillatory shear rheometer (Discovery HR-1, TA instrument) in parallel plate geometry (25 mm diameter). The measurements were carried out at a constant temperature ramp of 3° C./min between 25-100° C. with a rotational frequency of 1 Hz.

### Lap Shear Adhesion Test

Adhesion properties of the tested adhesive compositions were determined by using lap shear stress adhesion test according to ASTM F2255-05. Briefly, fresh poultry/porcine tissue pieces were cut into 20×45×1.5 mm pieces and hydrated with PBS. Adhesive compositions were cut into film pieces of 15×15×0.6 mm and carefully placed on the tissue. They were then topped with a 15×30 mm polyester hernia mesh strips, obtaining an overlapping area of 15×15 mm. Then, a small pressure was applied using a soldering station with a 15×15 mm square head (SLD soldering station model 936ESD) set to 70° C. and 95° C. The adhesive joints were left to solidify and were constantly hydrated with PBS prior to testing. The adhesive joints were pulled to failure (detachment) at a rate of 5 mm/min until the mesh and the substrate were separated, using an Instron machine with a 100 N load cell. The adhesive strength was determined by the load at maximum load divided by the initial contact area of the adhesive joint. Five replicates from each composition were tested and the mean adhesion strength and standard deviation around the mean were determined.

For time-dependent adhesion strength test, samples were stored in a sealed plastic bag with PBS placed in a water bath set to 37° C. for 1, 3, 5 and 8 hours, prior to testing in three replicates.

### Ball Burst Testing

Fresh porcine abdominal wall tissue was cut into approximately 16×16 cm squares. A 4 cm diameter punch was performed in the center and covered with an 8 cm diameter mesh. Adhesive films were cut into 15×15 mm and these were hot-melted using seven points of adhesion. For the burst test, the tissue was fixed on a 3D printed structure, designed specifically for the test. The burst strength was measured using an Instron machine with a 5 kN load cell set to compression mode and a cross speed of 300 mm/min. The burst strength was calculated from the overall overlapping area between the adhesive and the tissue. Five replicates of each composition were tested and the mean adhesion strength and standard deviation around the mean were determined.

### Example 1: Bioadhesive Compositions Having Two Components (Binary Compositions)

Bioadhesive compositions (blends) including the caffeic acid derivative, hydroxy caffeic acid (HCA) and PCL were prepared. First, the mixture was analyzed for its thermal behavior as it is one of hot-melt adhesives main significant characteristics.

Since high melting enthalpy polymers would need more energy for their melting process decreasing the melting temperature and melting enthalpy of the hot-melt adhesive is desired, as living tissues are temperature sensitive.

The influence of HCA on the thermal properties of mPCL are depicted in Table 1, below. mPCL neat showed an endothermic melting peak at 57° C., with a corresponding melting enthalpy of 61 J/gr. When HCA was blended with mPCL, the melting point temperature was reduced by 14%, in addition to a 24% reduction in the melting enthalpy value. This shows the hindrance effects of HCA on the crystallization of mPCL. Further presented in table 1, the values of degree of crystallinity (determined by the DSC and XRD analysis), which demonstrate the sensitivity of the adhesive compositions to mild heat.

TABLE 1
Melting temperature (Tₘ), melting enthalpy (ΔH) and degree
of crystallinity (DoC) calculated from DSC and XRD analysis.

| | Tₘ (° C.) | ΔH (gr/J) | DoC by DSC | DoC by XRD |
|---|---|---|---|---|
| mPCL | 57 | 61 | 45% | 57% |
| mPCL:HCA | 49 | 46 | 42% | 46% |

Next, the thermal characteristics of HCA alone were evaluated. As shown in the DSC Scan presented in FIG. 1, the melting temperature of HCA was approximately at 140° C., and its crystallization temperature was identified to be at 124° C. The XRD pattern of HCA is depicted in FIG. 2. The XRD pattern correlates with the DSC results, where the intense, sharp peaks indicates the crystalline form of HCA.

Despite the crystalline morphology of HCA, the absence of an endothermal peak in the DSC thermograms around the melting temperature of HCA (140° C.), implies that the HCA dissolves in the mPCL matrix. To further test this effect on solubility, a mPCL:HCA mixture (80:20) was also melt blended at 100° C., a temperature lower than the melting temperature of HCA. It would have been expected to identify a melting peak of the HCA at ±140° C. Surprisingly, DSC thermograms did not show an endothermal peak neither, supporting the notion that good molecular interactions exist between the two components.

Reference is now made to FIG. 3 which depicts the XRD patterns for mPCL (lower graph) compared to the binary blend of mPCL:HCA (upper graph). The results indicate the presence of three strong reflections in the mPCL phase at 2θ=21.4°, 22.1° and 23.6° where these correspond to the (110) (111) and (200) planes of orthorhombic crystal form, respectively. These strong reflections were also present in mPCL:HCA adhesive, suggesting that the addition of HCA to the polymer matrix did not change the crystal structure of the mPCL. However, a slight change in the peak width was noticed, where in XRD the width at half maximum of the peaks is usually related to the size of the crystallites, suggesting the formation of smaller spherulites. The absence of the characteristic diffraction peaks of HCA in the mPCL:HCA blend confirm the incorporation of HCA in the mPCL matrix.

Next, the adhesion strength of the bioadhesive composition was measured by performing lap shear adhesion test on fresh poultry breast tissue, as detailed above. The results are presented in FIG. 4. The adhesion strength of the adhesive composition was compared to mPCL neat (mPCL alone), to evaluate the role of HCA as an adhesion factor. Additionally, a cyanoacrylate-based adhesive (Dermabond®) was used for comparison. As shown in the results presented in FIG. 4, the mPCL:HCA mixture not only overcame the adhesion strength of mPCL neat, but was also exhibited up to 1.5 times higher adhesion strength as compared to the cyanoacrylate.

Thus, the results presented above clearly indicate the advantages of the binary hot-melt bioadhesive composition. The natural hydrophobicity of polycaprolactone compromise its adhesion strength, especially when implemented as soft tissues adhesives which require high adhesion strength in wet conditions. This hydrophobic nature makes it difficult for the PCL to create a strong adhesion with the polar functional groups encountered on living tissues. Nevertheless, as exemplified in the adhesion test results, the synergistic interaction with the hydrophilic catechol molecule (HCA), altered the hydrophobicity of the PCL, to create more functional sites which can interact with the living tissue.

### Example 2: Bioadhesive Compositions (Blends) Having Three Components (Tertiary Compositions)

The binary hot-melt compositions of Example 1, were further added with a copolymer poly(trimethylene carbonate-co-caprolactone) (coPTMC) at different ratios (10, 20 and 30 w/w percentage, where these rations are w/w between the coPTMC and the PCL (and not from the total composition with HCA), to form tertiary compositions. The inclusion of coPTMC was carried to try and obtain lower melting temperatures and lower melting enthalpies. The DSC thermograms of the tertiary blends are presented in FIG. 2. The blends were also compared to mPCL neat and the binary blend of mPCL:HCA. Surprisingly, even at high values of coPTMC, no significant decrease in the melting temperature was observed. However, while increasing coPTMC content, a significant decrease in the melting enthalpy was obtained and broader peak was noticed, suggesting a higher heterogeneity of the polymer crystals.

Next, the adhesion strength of the bioadhesive composition was measured by performing lap shear adhesion test on fresh poultry breast tissue. The results presented in FIG. 6, demonstrate that increasing the content of coPTMC in the adhesive composition does not improve the adhesion strength, and moreover, resulted in a decrease in the adhesion strength, as indicated by the arrow shown in FIG. 6. In addition, the compositions which included coPTMC exhibited adhesion failure between the adhesive and the poultry tissue.

The lower performance of the adhesive compositions containing coPTMC could be explained by the increase in the viscosity of the blend due to the high molecular weight of the copolymer, which even at low transition temperatures, the ability of the polymer to flow was compromised. FIG. 7 shows the complex viscosity of the hot-melt adhesive compositions, as a function of increasing temperature for different coPTMC amounts, indicating higher viscosity of these compositions. Thus, the inclusion of coPTMC in the adhesive compositions, to lower the activation temperature could have affected the adhesion strength due to an increase in the complex viscosity of the adhesive because of its high molecular weight, thus reducing substrate penetration.

### Example 3: Bioadhesive Compositions (Blends) Having Four Components (Quaternary Compositions)

The tertiary hot-melt compositions of Example 2, were further added with a low molecular weight polycaprolactone (PCL14K) at different ratios (20, 30 and 40% w/w) to form quaternary compositions.

For an adhesive composition to serve as a good adhesive, it should have both adhesion and cohesion strength. While the cohesion strength is mostly affected by the chemistry and properties of the components, the adhesion strength depends not only on the substrate tissue but also on its ability to flow and penetrate the tissue to create interlocking forces. The penetration of the adhesives can be controlled, inter alia, by adjusting the viscosity properties of the adhesives. To this aim, a low molecular weight polymer (polycaprolactone (PCL14K)) was introduced to the composition, to contribute to both properties and to lower the complex viscosity values of the adhesive composition and obtain a better wetting of the surface.

To this aim, a three-level full factorial design of experiments (DOE) was performed to obtain the optimum adhesive formulation (composition). The independent factors were PCL14K and coPTMC. The levels of these factors in the various compositions is shown in Table 2:

TABLE 2
Factor's level coding

| Main Effects | Factors | Level (−1) | Level (0) | Level (1) |
|---|---|---|---|---|
| A | PCL14K wt. % | 20% | 30% | 40% |
| B | PTMC wt. % | 10% | 20% | 30% |

The thermal properties of the different blends are presented in Error! Reference source not found. and includes the measured and modelled values for the compositions, for the melting enthalpy (ΔH). The residual average between both values was taken as the error value as shown in Equation 1.

TABLE 3
Full factorial design for ΔH

| | Sample | | | | | ΔH | |
|---|---|---|---|---|---|---|---|
| | A:B | PCL14K | coPTMC | Interaction | ΔH | [J/g] | |
| # | wt % | (A) | (B) | (AB) | [J/g] | DoE | Residual |
| 1 | 20:10 | −1 | −1 | 1 | 41 | 42 | 0.5 |
| 2 | 30:10 | 0 | −1 | 0 | 50 | 48 | 2.4 |
| 3 | 40:10 | 1 | −1 | −1 | 52 | 56 | 3.6 |
| 4 | 20:20 | −1 | 0 | 0 | 44 | 41 | 2.6 |
| 5 | 30:20 | 0 | 0 | 0 | 42 | 44 | 1.5 |
| 6 | 40:20 | 1 | 0 | 0 | 48 | 48 | 0.8 |
| 7 | 20:30 | −1 | 1 | −1 | 39 | 40 | 0.8 |
| 8 | 30:30 | 0 | 1 | 0 | 33 | 38 | 5.0 |
| 9 | 40:30 | 1 | 1 | 1 | 42 | 38 | 4.0 |
| | | | | | | Average | 2.4 |

Equation 1—Enthalpy calculation:

ΔH=44+3.1(A)+0.9(A)²−4.8(B)−0.6(B)²−3.9(AB)±2.4

The coPTMC first order effects and the interaction between both factors are significantly higher compared to the second order effects for both PCL14K and coPTMC factors. FIG. 5 illustrates the effects of the independent variables' levels using a surface response plot. The effects of PCL14K level are significant only at lower levels of coPTMC, while at higher levels of coPTMC, the effects of PCL14K are negligible, demonstrating the significance of the interaction effects between the two factors. All designated samples showed a decrease in the melting enthalpy compared to the enthalpy of pristine mPCL (61 J/gr), which could correlate to a decrease in the degree of crystallinity.

The complex viscosity values of the different samples are shown in Table 4. The lowest viscosity calculated value was obtained by Sample 40:10 showing the noteworthy effect of PCL14K on the adhesive flow. In addition, based on Equation 3 it was noticed that the second order effects and the interaction effects could be negligible compared to the first order effects of both factors. However, the first order effects of PCL14K had a greater significance effect on decreasing the complex viscosity rather than the first order effects of factor coPTMC as expected.

TABLE 4
Full factorial design for viscosity values at 90° C.

| | | | | | | Complex | |
|---|---|---|---|---|---|---|---|
| | Sample | | | | Complex | Viscosity | |
| | A:B | PCL14K | coPTMC | Interaction | Viscosity | DoE | |
| # | wt % | (A) | (B) | (AB) | [Pa · s] | [Pa · s] | Residual |
| 1 | 20:10 | −1 | −1 | 1 | 236 | 268 | 31.7 |
| 2 | 30:10 | 0 | −1 | 0 | 179 | 180 | 1.0 |
| 3 | 40:10 | 1 | −1 | −1 | 125 | 120 | 4.7 |
| 4 | 20:20 | −1 | 0 | 0 | 276 | 279 | 2.7 |
| 5 | 30:20 | 0 | 0 | 0 | 197 | 208 | 11.0 |
| 6 | 40:20 | 1 | 0 | 0 | 116 | 161 | 45.3 |
| 7 | 20:30 | −1 | 1 | −1 | 324 | 305 | 18.7 |
| 8 | 30:30 | 0 | 1 | 0 | 176 | 248 | 71.7 |
| 9 | 40:30 | 1 | 1 | 1 | 243 | 218 | 25.0 |
| | | | | | | Average | 24.0 |

### Equation 2

Viscosity₉₀=206−59(A)+14(A)²+34(B)+8(B)²+15(AB)±24

Error! Reference source not found. below shows the measured melting temperature and melting enthalpy of mPCL neat and two adhesive compositions. The incorporation of HCA to the mPCL matrix caused a significant decrease in both parameters. The 40:10 adhesive composition (sample no. 3 in Table 4) showed an additional decrease in the melting temperature, however a slight increase in the melting enthalpy was observed. As the 40:10 adhesive composition contains the highest level of PCL14K, an increase in the degree of crystallinity was expected, leading to a higher melting enthalpy. The combination of application temperature and low viscosity is crucial for hot-melt bio-adhesives as surface wetting is necessary to achieve good adhesiveness while tissue damage needs to be avoided. The thermal analysis results showed that coPTMC reduced the melting enthalpy of the polymer blends, while PCL14K increased it. However, PCL14K had a greater impact on the reduction of the complex viscosity at 90° C. (FIG. 9), which may increase the final adhesion strength. The composition named 40:10, which showed the lowest calculated complex viscosity was used for further experiments.

TABLE 5
Melting temperature measured from the first heating run
of pristine mPCL and two different adhesive compositions.

| | Melting | Melting |
|---|---|---|
| Sample | Temperature [° C.] | Enthalpy [J/g] |
| mPCL | 57 | 61 |
| mPCL:HCA | 49 | 46 |
| 40:10 composition | 46* | 52* |

*Measured value.

Next, the effects of the adhesion parameters of the selected composition (shown in Table 6, below) on the lap shear adhesion strength have been analyzed. The results are presented in FIG. 10 which compares the lap shear adhesion strength of different compositions. As shown, sample 40:10 exhibited the highest adhesion strength for both the first and the second tests on fresh poultry tissue. This can be attributed to the addition of PCL14K which lowers the complex viscosity of the adhesive, as shown in Table 4. Although sample 40:10 exhibited the highest melting enthalpy (56 J/gr), this value could be enough to accomplish a reduction in the application time from 20 seconds to 5 seconds. Furthermore, the maximum temperature measured on the tissue during the adhesive application was 47±3.0.

TABLE 6
Adhesion parameters of the selected composition

| | Application | | |
|---|---|---|---|
| | Temperature | Application time | Max. Temperature |
| | [° C.] | [s] | on the tissue [° C.] |
| First Run | 70 | 20 | 42 ± 2.0 |
| Second Run | 95 | 5 | 47 ± 3.0 |

Next, Ex-vivo lap shear adhesion test was performed on fresh porcine abdominal wall tissue, as it has higher similarity with human tissue. The results are presented in FIG. 8, which depicts the adhesion strength of the 40:10 adhesive composition, as compared to cyanoacrylate-based glue (Dermabond®) and mPCL neat. As shown, composition 40:10 reached an adhesion strength at least similar to the cyanoacrylate-based glue.

In addition, the adhesion strength of the composition was analyzed as a function of time, as detailed above. The results are presented in FIG. 12. As shown in FIG. 12, there is an initial decrease in the adhesion strength flowing 1 hour after the application of the adhesive composition. However, following the first hour, there is a constant increase in the adhesion strength of the adhesive, reaching a maximum strength of 7.5 kPa after eight hours after the adhesive was applied. The samples were stored in PBS at 37° C. (body temperature), indicating the HCA indeed improves the adhesive strength between the hernia mesh placed on the adhesive film, and the porcine tissue.

Additionally, the burst strength of the adhesive composition 40:10 (DOE 40:10) was compared to the burst strength of a cyanoacrylate adhesive. The results are shown in FIG. 13. As shown, the burst strength achieved by the 40:10 composition correlates with the cyanoacrylate adhesive burst strength, exhibiting good mechanical properties.

### REFERENCES

[1] A. Kingsnorth, Treating inguinal hernias, BMJ. 328 (2004) 59-60. doi:10.1136/bmj.328.7431.59.
[2] L. M. Funk, K. A. Perry, V. K. Narula, D. J. Mikami, W. S. Melvin, Current national practice patterns for inpatient management of ventral abdominal wall hernia in the United States, Surg. Endosc. 27 (2013) 4104-4112. doi:10.1007/s00464-013-3075-4.
[3] A. Grant, P. Go, A. Fingerhut, A. Kingsnorth, J. Merello, P. O'Dwyer, J. Payne, Repair of Groin Hernia With Synthetic Mesh, Ann. Surg. 235 (2002) 322-332. doi:10.1097/00000658-200203000-00003.
[4] M. T. Nguyen, R. L. Berger, S. C. Hicks, J. A. Davila, L. T. Li, L. S. Kao, M. K. Liang, Comparison of outcomes of synthetic mesh vs suture repair of elective primary ventral herniorrhaphy: A systematic review and meta-analysis, JAMA Surg. 149 (2014) 415-421. doi:10.1001/jamasurg.2013.5014.
[5] A. S. Poobalan, J. Bruce, W. C. S. Smith, P. M. King, Z. H. Krukowski, W. A. Chambers, A review of chronic pain after inguinal herniorrhaphy, Clin. J. Pain. 19 (2003) 48-54.
[6] C. A. Courtney, K. Duffy, M. G. Serpell, P. J. O'Dwyer, Outcome of patients with severe chronic pain following repair of groin hernia, Br. J. Surg. 89 (2002) 1310-1314. doi:10.1046/j.1365-2168.2002.02206.x.
[7] V. Bhagat, M. L. Becker, Degradable Adhesives for Surgery and Tissue Engineering, Biomacromolecules. 18 (2017) 3009-3039. doi:10.1021/acs.biomac.7b00969.
[8] H. J. Cha, D. S. Hwang, S. Lim, Development of bioadhesives from marine mussels, Biotechnol. J. 3 (2008) 631-638. doi:10.1002/biot.200700258.
[9] J. L. Murphy, L. Vollenweider, F. Xu, B. P. Lee, Adhesive performance of biomimetic adhesive-coated biologic scaffolds, Biomacromolecules. 11 (2010) 2976-84. doi:10.1021/bm1007794.
[10] Y. Liu, H. Meng, S. Konst, R. Sarmiento, R. Rajachar, B. P. Lee, Injectable Dopamine-Modified Poly(ethylene glycol) Nanocomposite Hydrogel with Enhanced Adhesive Property and Bioactivity, ACS Appl. Mater. Interfaces. 6 (2014) 16982-16992. doi:10.1021/am504566v.
[11] K. A. Burke, D.C. Roberts, D. L. Kaplan, Silk Fibroin Aqueous-Based Adhesives Inspired by Mussel Adhesive Proteins, Biomacromolecules. 17 (2015) 237-245. doi:10.1021/acs.biomac.5b01330.
[12] J. H. Ryu, S. Hong, H. Lee, Bio-inspired adhesive catechol-conjugated chitosan for biomedical applications: A mini review, Acta Biomater. 27 (2015) 101-115. doi:10.1016/J.ACTBIO.2015.08.043.
[13] C. Fan, J. Fu, W. Zhu, D.-A. Wang, A mussel-inspired double-crosslinked tissue adhesive intended for internal medical use, Acta Biomater. 33 (2016) 51-63. doi:10.1016/J.ACTBIO.2016.02.003.
[14] H. Zhang, L.'Igia Bré, B. Bré, T. Zhao, B. Newland, M. Da Costa, W. Wang, A biomimetic hyperbranched poly(amino ester)-based nanocomposite as a tunable bone adhesive for sternal closure, J. Mater. Chem. B. 2 (2014) 4067-4071|. doi:10.1039/c4tb00155a.
[15] H. Zhang, L. P. Bré, T. Zhao, Y. Zheng, B. Newland, W. Wang, Mussel-inspired hyperbranched poly(amino ester) polymer as strong wet tissue adhesive, Biomaterials. 35 (2014) 711-719. doi:10.1016/J.BIOMATERIALS.2013.10.017.
[16] D. Ruiz-Molina, J. S. Poseu, F. Busque, F. Nador, J. Mancebo, J. Saiz-Poseu, J. Mancebo-Aracil, F. Nador, F. Busqué, D. Ruiz-Molina, Titel: The Chemistry behind Catechol-Based Adhesion The Chemistry behind Catechol-Based Adhesion, Angew. Chem. Int. Ed. Angew. Chem. 10 (2018). doi:10.1002/ange.201801063.
[17] G. P. Maier, C. M. Bernt, A. Butler, Catechol oxidation: considerations in the design of wet adhesive materials, Biomater. Sci. 6 (2018) 332-339. doi:10.1039/C7BM00884H.
[18] S. Ahmad Mian, S. U. Azzam, G. Rahman, E. Ahmed, Density Functional Theory Study of Mussel Adhesive Protein (L-Dopa and Catechol) Cross-Linking, MedCrave. 1 (2017). doi:10.15406/mojboc.2017.01.00037.
[19] P. Kord Forooshani, B. P. Lee, Recent approaches in designing bioadhesive materials inspired by mussel adhesive protein, J. Polym. Sci. Part A Polym. Chem. 55 (2017) 9-33. doi:10.1002/pola.28368.
[20] W. Y. Choi, C. M. Lee, H. J. Park, Development of biodegradable hot-melt adhesive based on poly-ε-caprolactone and soy protein isolate for food packaging system, LWT—Food Sci. Technol. 39 (2006) 591-597. doi:10.1016/J.LWT.2005.04.012.
[21] S. Schaible, C. Bernet, P. Ledergeber, T. Balmer, C. Brändli, Basic Study on the Evaluation of Thermoplastic Polymers as Hot-Melt Adhesives for Mixed-Substrate Joining, Open J. Appl. Sci. 6 (2016) 579-592. doi:10.4236/ojapps.2016.68057.
[22] R. C. Neuman, Experimental strategies for polymer scientists and plastics engineers, Henser Publishers, Munich, 1997.
[23] H. Kweon, M. K. Yoo, I. K. Park, T. H. Kim, H. C. Lee, H.-S. Lee, J.-S. Oh, T. Akaike, C.-S. Cho, A novel degradable polycaprolactone networks for tissue engineering, Biomaterials. 24 (2003) 801-808. doi:10.1016/S0142-9612(02)00370-8.
[24] J.-P. Dumas, P. Tordjeman, Y. Zeraouli, F. Di Paolo, J. Dumas, Heat transfer model for the cooling of hot melt adhesives, J. Adhes. Sci. Technol. 12 (1998) 399-413. doi:10.1163/156856198X00119.
[25] N. Ri Im, K. Mi Kim, S. Ji Young, S. Nam Park, Physical characteristics and in vitro skin permeation of elastic liposomes loaded with caffeic acid-hydroxypropyl-β-cyclodextrin, Korean J. Chem. Eng. 33 (2016) 2738-2746. doi:10.1007/s11814-016-0146-y.
[26] G. C. Alfonso, P. Russell, Kinetics of Crystallization in Semicrystalline/Amorphous Polymer Mixtures, Macromolecules. 19 (1986) 1143-1152. doi:10.1021/ma00158a036.
[27] E.-C. Chen, T.-M. Wu, Isothermal crystallization kinetics and thermal behavior of poly(3-caprolactone)/multi-walled carbon nanotube composites, Polym. Degrad. Stab. 29 (2007) 1009-1015. doi:10.1016/j.polymdegradstab.2007.02.019.
[28] E. Sanchez-Rexach, E. Meaurio, J. Iturri, J. L. Toca-Herrera, S. Nir, M. Reches, J. R. Sarasua, Miscibility, interactions and antimicrobial activity of poly(ε-caprolactone)/chloramphenicol blends, Eur. Polym. J. 102 (2018) 30-37. doi:10.1016/j.eurpolymj.2018.03.011.
[29] W. Wang, G. Caetano, W. S. Ambler, J. J. Blaker, M. A. Frade, P. Mandal, C. Diver, P. Bártolo, Enhancing the Hydrophilicity and Cell Attachment of 3D Printed PCL/Graphene Scaffolds for Bone Tissue Engineering, Mater. 9 (2016) 992. doi:10.3390/ma9120992.
[30] A. P. Pêgo, A. A. Poot, D. W. Grijpma, J. Feijen, In vitro degradation of trimethylene carbonate based (Co)polymers, Macromol. Biosci. 2 (2002) 411-419. doi:10.1002/mabi.200290000.
[31] a P. Pêgo, a Poot, D. W. Grijpma, J. Feijen, Copolymers of trimethylene carbonate and epsilon-caprolactone for porous nerve guides: synthesis and properties, J. Biomater. Sci. Polym. Ed. 12 (2001) 35-53. doi:10.1163/156856201744434.
[32] O. Pinkas, D. Goder, R. Noyvirt, S. Peleg, M. Kahlon, M. Zilberman, Structuring of composite hydrogel bioadhesives and its effect on properties and bonding mechanism, Acta Biomater. 51 (2017) 125-137. doi:10.1016/j.actbio.2017.01.047.
[33] W. Li, L. Bouzidi, S. S. Narine, Current Research and Development Status and Prospect of Hot-Melt Adhesives: A Review, Ind. Eng. Chem. Res. 47 (2008) 7524-7532. doi:10.1021/ie800189b.
[34] V. Crescenzi, G. Manzini, G. Calzolari, C. Borri, Thermodynamics of fusion of poly-β-propiolactone and poly-ϵ-caprolactone. comparative analysis of the melting of aliphatic polylactone and polyester chains, Eur. Polym. J. 8 (1972) 449-463.
[35] A. S. Luyt, S. Gasmi, Influence of blending and blend morphology on the thermal properties and crystallization behavior of PLA and PCL in PLA/PCL blends, J. Mater. Sci. 51 (2016) 4670-4681. doi:10.1007/s10853-016-9784-z.
[36] V. Speranza, A. Sorrentino, F. De Santis, R. Pantani, Characterization of the polycaprolactone melt crystallization: complementary optical microscopy, DSC, and AFM studies, ScientificWorldJournal. 2014 (2014) 720157. doi:10.1155/2014/720157.
[37] C. R. Deeken, S. P. Lake, Mechanical properties of the abdominal wall and biomaterials utilized for hernia repair, J. Mech. Behav. Biomed. Mater. 74 (2017) 411-427. doi:10.1016/J.JMBBM.2017.05.008.

1.-32. (canceled)

33. A bioadhesive composition comprising at least one caffeic acid derivative and at least one synthetic thermoplastic polymer, wherein exposure to heat causes the bioadhesive composition to transform into a non-solid state and to cohesively adhere to a biological tissue upon subsequent cooling thereof.

34. The bioadhesive composition according to claim 33, wherein the synthetic thermoplastic polymer comprise polycaprolactone.

35. The bioadhesive composition according to claim 33, wherein the polycaprolactone comprises medium weight polycaprolactone.

36. The bioadhesive composition according to claim 33, wherein the polycaprolactone has a molecular weight of between about 20,000 Da to 90,000 Da.

37. The bioadhesive composition according to claim 33, comprising at least about 15% (w/w) caffeic acid derivative of the total composition.

38. The bioadhesive composition according to claim 33, wherein the caffeic acid derivative is hydroxy caffeic acid (HCA).

39. The bioadhesive composition according to claim 33, wherein the caffeic acid derivative does not include an amine group.

40. The bioadhesive composition according to claim 33, further comprising copolymer poly(trimethylene-co-polycaprolactone) (coPTMC).

41. The bioadhesive composition according to claim 33, further comprising polycaprolactone having a molecular weight in the range of about 5000-19,000 Da.

42. The bioadhesive composition according to claim 33, transforming into a non-solid state at a temperature of over about 40° C.

43. The bioadhesive composition according to claim 33, transforming into a non-solid state at a temperature of between 40° C. and 120° C.

44. The bioadhesive composition according to claim 33, in the form of a solid state film.

45. A method for producing the bioadhesive composition of claim 33, the method comprising mixing the caffeic acid derivative and the at least one synthetic thermoplastic polymer to form homogenous mixture while heating to a melting temperature.

46. The method according to claim 45, wherein the melting temperature is over about 100° C.

47. The method according to claim 45, further comprising a step of cooling the composition to a solid state.

48. The method according to claim 45, further comprising a step of compression molding the composition to form a solid state film.

49. The bioadhesive composition of claim 33, for use in adhering a substrate/matrix to a tissue.

50. The bioadhesive composition for use according to claim 49 wherein the matrix is selected from a mesh, gauze, dressing, bandage, and graft.

51. The bioadhesive composition for use according to claim 49, wherein the mesh is a hernia mesh.

52. A kit comprising a substrate and the bioadhesive composition according to claim 33, wherein at least a portion of the substrate is coated, impregnated or formed with the bioadhesive composition, wherein the substrate is selected from a mesh, a gauze, a dressing, a bandage, a graft, or combinations thereof.
